SEQUENCE LISTING (12 sequences)

SEQ ID NO: 1 - protein, 8 aa, Homo sapiens
  MOD_RES (4)..(4): METHYLATION
ARTKQTAR

SEQ ID NO: 2 - protein, 5 aa, Homo sapiens
  MOD_RES (3)..(3): METHYLATION
SGRGK

SEQ ID NO: 3 - protein, 5 aa, Homo sapiens
  MOD_RES (5)..(5): ACETYLATION
SGRGK

SEQ ID NO: 4 - protein, 5 aa, Homo sapiens
  MOD_RES (3)..(3): METHYLATION
SGRGK

SEQ ID NO: 5 - protein, 5 aa, Homo sapiens
  MOD_RES (1)..(1): PHOSPHORYLATION
SGRGK

SEQ ID NO: 6 - protein, 9 aa, Tetrahymena thermophila
  MOD_RES (5)..(5): METHYLATION
QTARKSTGV

SEQ ID NO: 7 - protein, 11 aa, Homo sapiens
  MOD_RES (9)..(9): Phosphorylation
SAPAPKKGSKK

SEQ ID NO: 8 - protein, 9 aa, Homo sapiens
  MOD_RES (5)..(5): methylation
QTARKSTGG

SEQ ID NO: 9 - protein, 7 aa, Homo sapiens
  MOD_RES (4)..(4): methylation
AARKSAP

SEQ ID NO: 10 - protein, 10 aa, Homo sapiens
  MOD_RES (4)..(4): methylation
AARKSAPVCG

SEQ ID NO: 11 - protein, 11 aa, Homo sapiens
  MOD_RES (5)..(5): methylation
SGGVKKPHKCG

SEQ ID NO: 12 - protein, 10 aa, Homo sapiens
  MOD_RES (4)..(4): methylation
RHRKILRDCG

(19) United States
(12) Patent Application Publication
(10) Pub. No.: US 2005/0069931 A1
(43) Pub. Date: Mar. 31, 2005

(54) Non-invasive diagnostic test utilizing histone modification markers

(76) Inventors: C. David Allis, New York, NY (US); David E. Bruns, Greenwood, VA (US); Alan H. Drummond, Oxon (GB)

Correspondence Address: Schwegman, Lundberg, Woessner & Kluth, P.A., P.O. Box 2938, Minneapolis, MN, 55402, US

(21) Appl. No.: 10/922,806

(22) Filed: Aug. 19, 2004

Related U.S. Application Data
(63) Continuation of application No. PCT/US03/04661, filed on Feb. 19, 2003.
(60) Provisional application No. 60/358,325, filed on Feb. 20, 2002.
(60) Provisional application No. 60/365,459, filed on Mar. 19, 2002.

(51) Int. Cl. C12Q 1/68; C12P 19/34

(52) U.S. Cl. 435/6; 435/91.2

(57) ABSTRACT

The present invention relates to the use of antibodies directed against specific histone amino terminus modifications as diagnostic indicators of disease or congenital defects. In one embodiment, nucleosomes are isolated from a blood or serum sample of a patient using histone specific antibodies and the accompanying DNA is purified and analyzed for diagnostic and screening purposes.

### RELATED APPLICATIONS

This application claims priority under 35 U.S.C. §119(e) to provisional patent application Nos. 60/358,325, filed Feb. 20, 2002 and 60/365,459, filed Mar. 19, 2002 the disclosures of which are incorporated herein by reference in their entirety.

### US GOVERNMENT RIGHTS

This invention was made with United States Government support under Grant Nos. GM 40922 and GM 53512, awarded by the National Institutes of Health. The United States Government has certain rights in the invention.

### FIELD OF THE INVENTION

The present invention is directed to compositions and methods for diagnosing various disease states. More particularly, the method uses antibodies that are specific for unique histone epitopes, created by post-translational modification of histone proteins, to isolate cell-free nucleosomes from an individual's blood, plasma or serum.

### BACKGROUND OF THE INVENTION

In eukaryotes, DNA is complexed with histone proteins to form nucleosomes, the repeating subunits of chromatin. This packaging of DNA imposes a severe restriction to proteins seeking access to DNA for DNA-templated processes such as transcription or replication. It is becoming increasingly clear that post-translational modifications of histone amino-termini play an important role in determining the chromatin structure of the eukaryotic cell genome as well as regulating the expression of cellular genes.

A large number of covalent modifications of histones have been documented, including acetylation, phosphorylation, methylation, ubiquitination, and ADP ribosylation, that take place on the amino terminus “tail” domains of histones. Such diversity in the types of modifications and the remarkable specificity for residues undergoing these modifications suggest a complex hierarchy of order and combinatorial function that remains unclear. Of the covalent modifications known to take place on histone amino-termini, acetylation is perhaps the best studied and appreciated. Recent studies have identified previously characterized coactivators and corepressors that acetylate or deacetylate, respectively, specific lysine residues in histones in response to their recruitment to target promoters in chromatin (See Berger (1999) Curr. Opin. Genet. Dev. 11, 336-341). These studies provide compelling evidence that chromatin remodeling plays a fundamental role in the regulation of transcription from nucleosomal templates.

Through the use of antibodies that specifically recognize histones bearing specific post-translational modifications, applicants have been elucidating a “histone code.” In particular, evidence is emerging that histone proteins, and their associated covalent modifications, contribute to a mechanism that can alter chromatin structure, thereby leading to inherited differences in transcriptional “on-off” states or to the stable propagation of chromosomes by defining a specialized higher-order structure. Thus these specific modifications can serve as markers that indicate the transcriptional status of the associated DNA.

It has been reported recently that nucleosomes can be detected in the serum of healthy individuals (Stroun et al., Annals of the New York Academy of Sciences 906:161-168 (2000)) as well as individuals afflicted with a disease state. Moreover, it has been reported that the serum concentration of nucleosomes is considerably higher in patients suffering from benign and malignant diseases (Holdenrieder et al., Int J Cancer, 95 (2): 114-120 (Mar. 20, 2001)). Presumably, the high concentration of nucleosomes in tumor bearing patients derives from apoptosis, which occurs spontaneously in proliferating tumors. Thus, the presence of elevated levels of nucleosomes in the blood of patients can serve as a diagnostic of diseases associated with enhanced cell death (Holdenrieder et al., Anticancer Res, 19 (4A): 2721-2724 (1999)).

Prior to the present invention investigators simply monitored the total number of nucleosomes present in an individual's blood without characterizing the histone types comprising the detected nucleosomes. Nucleosomes circulating in the blood are anticipated to contain uniquely modified histones, wherein the unique histone epitope and/or the associated DNA can be correlated with a particular disease state. Accordingly, one aspect of the present invention is directed to the identification of cell-free mono or oligonucleosomes through the use of antibodies that specifically bind to modified histone proteins. The identification of such modified histones can serve as diagnostic markers of disease and congenital defects.

### SUMMARY OF THE INVENTION

The present invention is directed to a non-invasive diagnostic method for detecting nucleosomes present in an individual's bodily fluid, wherein the nucleosomes comprise one or more modified histones. More particularly, antibodies have been generated against specific post-translational modifications of the amino terminus of histones and these antibodies are used to detect cell-free nucleosome that contain a preselected modified histone. Alterations in the overall number and/or ratio of the different types of modified histones can be used for diagnostic purposes. In addition, the type of modified histone that is associated with a particular nucleic acid sequence can used as a diagnostic of a disease state.

### BRIEF DESCRIPTION OF THE DRAWINGS

FIG. 1 is a diagram representing post-translational modifications found on the amino terminus of human histone proteins H2A, H2B, H3 and H4.

### DETAILED DESCRIPTION OF THE INVENTION

Definitions

In describing and claiming the invention, the following terminology will be used in accordance with the definitions set forth below.

As used herein, the term “nucleic acid” encompasses RNA as well as single and double-stranded DNA and cDNA. Furthermore, the terms, “nucleic acid,” “DNA,” “RNA” and similar terms also include nucleic acid analogs, i.e. analogs having other than a phosphodiester backbone. For example, the so-called “peptide nucleic acids,” which are known in the art and have peptide bonds instead of phosphodiester bonds in the backbone, are considered within the scope of the present invention.

As used herein, the terms “complementary” or “complementarity” are used in reference to polynucleotides (i.e., a sequence of nucleotides) related by the base-pairing rules. For example, for the sequence “A-G-T,” is complementary to the sequence “T-C-A.”

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the length of the formed hybrid, and the G:C ratio within the nucleic acids.

The term “peptide” encompasses a sequence of 3 or more amino acids wherein the amino acids are naturally occurring or synthetic (non-naturally occurring) amino acids. Peptide mimetics include peptides having one or more of the following modifications:
1. peptides wherein one or more of the peptidyl —C(O)NR— linkages (bonds) have been replaced by a non-peptidyl linkage such as a —CH2-carbamate linkage (—CH₂OC(O)NR—), a phosphonate linkage, a —CH₂-sulfonamide (—CH₂—S(O)₂NR—) linkage, a urea (—NHC(O)NH—) linkage, a —CH₂-secondary amine linkage, or with an alkylated peptidyl linkage (—C(O)NR—) wherein R is C₁-C₄ alkyl;
2. peptides wherein the N-terminus is derivatized to a —NRR₁ group, to a —NRC(O)R group, to a —NRC(O)OR group, to a —NRS(O)₂R group, to a —NHC(O)NHR group where R and R₁ are hydrogen or C₁-C₄ alkyl with the proviso that R and R₁ are not both hydrogen;
3. peptides wherein the C terminus is derivatized to —C(O)R₂ where R₂ is selected from the group consisting of C₁-C₄ alkoxy, and —NR₃R₄ where R₃ and R₄ are independently selected from the group consisting of hydrogen and C₁-C₄ alkyl.

Naturally occurring amino acid residues in peptides are abbreviated as recommended by the IUPAC-IUB Biochemical Nomenclature Commission as follows: Phenylalanine is Phe or F; Leucine is Leu or L; Isoleucine is Ile or I; Methionine is Met or M; Norleucine is Nle; Valine is Vat or V; Serine is Ser or S; Proline is Pro or P; Threonine is Thr or T; Alanine is Ala or A; Tyrosine is Tyr or Y; Histidine is His or H; Glutamine is Gln or Q; Asparagine is Asn or N; Lysine is Lys or K; Aspartic Acid is Asp or D; Glutamic Acid is Glu or E; Cysteine is Cys or C; Tryptophan is Trp or W; Arginine is Arg or R; Glycine is Gly or G, and X is any amino acid. Other naturally occurring amino acids include, by way of example, 4-hydroxyproline, 5-hydroxylysine, and the like.

As used herein, the term “conservative amino acid substitution” is defined herein as exchanges within one of the following five groups:
I. Small aliphatic, nonpolar or slightly polar residues:
   Ala, Ser, Thr, Pro, Gly;
II. Polar, negatively charged residues and their amides:
   Asp, Asn, Glu, Gln;
III. Polar, positively charged residues:
   His, Arg, Lys;
IV. Large, aliphatic, nonpolar residues:
   Met Leu, Ile, Val, Cys
V. Large, aromatic residues:
   Phe, Tyr, Trp

As used herein, the term “purified” and like terms relate to the isolation of a molecule or compound in a form that is substantially free (at least 60% free, preferably 75% free, and most preferably 90% free) from other components normally associated with the molecule or compound in a native environment.

The term “disease state” is intended to encompass any condition that is associated with an impairment of the normal state of a living animal or plant including congenital defects, pathological conditions such as cancer, and responses to environmental factors and infectious agents (bacterial, viral, etc.).

“Therapeutic agent,” “pharmaceutical agent” or “drug” refers to any therapeutic or prophylactic agent which may be used in the treatment (including the prevention, diagnosis, alleviation, or cure) of a malady, affliction, disease or injury in a patient.

As used herein, the term “treating” includes alleviating the symptoms associated with a specific disorder or condition and/or preventing or eliminating said symptoms. For example, treating cancer includes preventing or slowing the growth and/or division of cancer cells as well as killing cancer cells.

As used herein, the term “pharmaceutically acceptable carrier” encompasses any of the standard pharmaceutical carriers, such as a phosphate buffered saline solution, water and emulsions such as an oil/water or water/oil emulsion, and various types of wetting agents.

As used herein, the term “antibody” refers to a polyclonal or monoclonal antibody or a binding fragment thereof such as Fab, F(ab′)₂ and Fv fragments.

As used herein, the term “parenteral” includes administration subcutaneously, intravenously or intramuscularly.

As used herein, the term “modified histone” refers to a histone protein selected from the group consisting of H2A, H2B, H3 and H4, wherein one or more of the last 30 amino acid residues of the amino terminus have been modified post-translationally through acetylation, methylation, phosphorylation or ubiqutination.

The term “modified amino acid” as used herein includes an amino acid residue comprising one or more modifying groups covalently bound to the amino acid. For example, each modified lysine residue has the capacity to be mono-, di-, or tri-methylated, and the general reference to a methylated lysine is intended to encompass all three of these possibilities.

As used herein, the term “active gene sequence” refers to a gene that is competent for transcriptional activity.

As used herein, the term “inactive gene sequence” refers to a gene sequence that is not competent for transcriptional activity.

The Invention

It has recently been reported that nucleosomes can be detected in the blood of patients and that elevated blood level concentrations of nucleosomes may serve as a diagnostic of cancer. However, these previous studies simply monitored total nucleosome populations and failed to account for subpopulations of nucleosomes that differ from each other based on histone content. Applicants have discovered that specific post-translational modification of histones contribute to a mechanism that can alter chromatin structure, and this chromatin remodeling is believed to play a fundamental role in the regulation of transcription from nucleosomal templates. Furthermore, applicants are the first to demonstrate that nucleosomes retain their post-translational modifications during the apoptotic process. In particular, as described in Example 1, nucleosomes from breast tumor cells that have undergone apoptosis retain their methyl, phosphorylation and acetyl modifications, and such modifications can be detected using antibodies raised against these specific modifications.

The present invention is directed to an improved diagnostic test for disease that involves screening a warn blooded animal's blood for the presence of elevated cell-fee modified nucleosome populations or altered chromatin structures. More particularly, the present invention is directed to the use of antibodies that bind to specific post-translational modifications of the amino or carboxy terminus of histone peptides. The presence of unique histone modifications on detected nucleosomes provides information regarding chromatin structure and the transcriptional activity of nucleic acid sequences associated with the modified histone proteins.

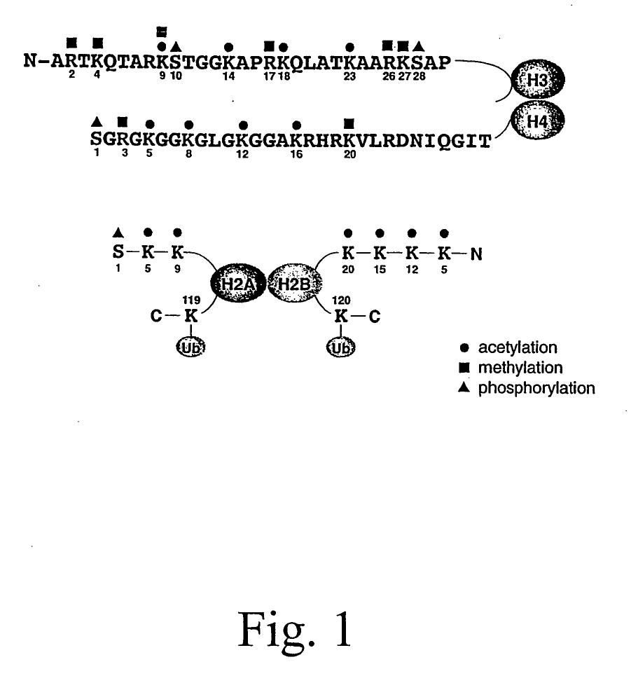

Accordingly, the present invention is directed to an improve diagnostic screen that uses antibodies, that are specific for unique epitopes formed by post-translational modifications on the flexible N-terminal and C-terminal tails of the core histone proteins, to isolate nucleosomes from the blood (and other bodily fluids) of mamnials, more preferably from humans. Suitable post-translational modifications of histone amino acid residues that serve as unique epitopes for use in the present invention are described in FIG. 1. The detection of one or more specific histone modifications in the blood of an individual may serve as a diagnostic for a particular disease state.

In accordance with one embodiment of the invention, antibodies directed to unique histone markers that are associated with active gene sequences (euchromatin) or inactive gene sequences (heterochromatin) can be used to detect inappropriate gene expression that is indicative of a disease state. For example, screening the nucleosome population present in an individual's blood or other bodily fluid may reveal the inactivation of a tumor suppression gene or alternatively the activation of an oncogene.

The method of detecting such active or inactive gene sequences in an individual comprises the steps of obtaining a body fluid sample from the individual and isolating nucleosomes from that sample using a modified histone specific antibody. The nucleosomes can be recovered from one or more bodily fluids of a patient including urine, blood, lymph, plasma or serum, thus providing a minimally invasive screen for diagnosing disease states. In one embodiment, the nucleosomes are recovered from the blood, plasma or serum of an individual. To enhance the stability of nucleosomes present in the bodily fluids, the samples are preferably treated with 10 mM EDTA and stored at a temperature of −20 degrees C. As a precursor to immunoprecipitation with antibodies that target modified histones, nucleosomes in blood, plasma or serum can be first concentrated by collection on poly-lysine- or streptavidin-coated solid supports. The latter approach utilizes the biotinyltransferase activity present in blood (Hymes & Wolf, J. Nutr. 129, 485S-489S, 1999) to biotinylate histones preferentially prior to capture on streptavidin.

The anti-modified histone antibodies used in the present invention can be selected from any of the antibodies that target known histone epitopes formed by post-translational modification of the histone tails. A list of several post-translational modifications of histone tails that can serve as epitopes for the antibodies used in the present invention is provided in FIG. 1. Each of these antibodies can be used to isolate nucleosomes present in a patient's blood that contain the relevant modified histone. The identification of the modified histone in the blood may be indicative of a particular disease or disorder. A significant increase (relative to wild type levels) in the number of cell-free nucleosomes detected by these antibodies in an individual, and/or an alteration in the ratio of one or more particular histone modification relative to another histone modification, may indicate a particular disease state.

In one embodiment an antibody is selected that binds to a modified histone known to be associated with active gene sequences or alternatively binds to a modified histone associated with inactive gene sequences. Histone epitopes that have been identified as being associated with gene activation include the following:

| | |
|---|---|
| Ala Arg Thr Lys(M) Gln Thr Ala Arg, | (SEQ ID NO: 1) |
| Ser Gly Arg(M) Gly Lys, | (SEQ ID NO: 2) |
| Ser Gly Arg Gly Lys(A), | (SEQ ID NO: 3) |
| Ser Gly Arg(M) Gly Lys(A), | (SEQ ID NO: 4) |
| and | |
| Ser(P) Gly Arg(M) Gly Lys(A), | (SEQ ID NO: 5) |

wherein “Ser(P), “Arg(M)” and “Lys(A)” represents the modified amino acids phosphorylated serine, methylated arginine and acetylated lysine, respectively. In one embodiment the antibody is specific for a peptide sequence comprising SEQ ID NO: 1 wherein the lysine residue is dimethylated. Histone epitopes that have been identified as being associated with gene inactivation include the following:

| | |
|---|---|
| Gln Thr Ala Arg Lys(M) Ser Thr Gly | (SEQ ID NO: 6) |
| Val | |
| Gln Thr Ala Arg Lys(M) Ser Thr Gly | (SEQ ID NO: 8) |
| Gly | |
| Ala Ala Arg Lys(M) Ser Ala Pro. | (SEQ ID NO: 9) |

In one embodiment the antibody is specific for a peptide sequence comprising SEQ ID NO: 8 wherein the lysine residue is dimethylated. These antibodies can be used to detect any abnormal gene expression that would indicate a disease state. Alternatively, the nucleic acid sequences associated with the nucleosomes (isolated from the blood by immunoprecipitation using one of the histone modification specific antibodies) can be analyzed using standard techniques to help diagnose a disease state, or the potential for disease (i.e. identification of latent viruses or other genetic precondition).

In accordance with one embodiment of the present invention the nucleosomes from an individual's body fluid are isolated by immunoprecipitation using one or more of the modified histone specific antibodies of the present invention. Alternatively, modified histone specific antibodies of the present invention can be linked to an insoluble support to provide a means of isolating cell-free nucleosomes from a sample. The support may be in particulate or solid form and could include, but is not limited to: a plate, a test tube, beads, a ball, a filter or a membrane. Methods for fixing antibodies to insoluble supports are known to those skilled in the art. In one embodiment an antibody of the current invention is fixed to an insoluble support that is suitable for use in affinity chromatography. After the sample has been contacted with the modified histone specific antibodies under conditions suitable to allow specific binding of the antibody to its target antigen, nucleosomes comprising the modified histones can be isolated using standard techniques known to hose skilled in the art.

Once the nucleosomes have been isolated from the sample, the DNA associated with the nucleosomes can be recovered using standard techniques, including optionally amplifying the recovered DNA through PCR or other amplification techniques. In accordance with one embodiment the DNA associated with the inimunoprecipitated nucleosomes is purified and the genes encoded by that DNA are identified. Depending on the specific antibody used to initially isolate the nucleosomes from the bodily fluid sample, this procedure allows one to identify genes that are either active or inactive in the individual.

The steps used to identify the genes encoded by the DNA associated with the isolated nucleosomes can include any of the analytical procedures known to those skilled in the art. In accordance with one embodiment the gene sequences are identified by direct microsequencing the purified DNA. Alternatively, in one embodiment the purified DNA is first amplified using PCR technology or other amplifying technique before further analysis of the DNA, such as sequence analysis.

In one embodiment the genes encoded by the DNA associated with the isolated nucleosomes can be identified by contacting the purified DNA with known nucleic acid sequences under conditions suitable for hybridization of complementary sequences, wherein hybridization of the purified DNA to its complement identifies the gene. For example, Southern Blots analysis can be conducted wherein either the known DNA sequences or the purified DNA serves as the labeled probe, and the unlabeled sequences are immobilized on a solid surface.

The nucleic acid probes can be labeled with a detectable marker using standard techniques known to those skilled in the art, and it is not intended that the present invention be limited to any particular detection system or label. For example the nucleic acid probes can be labeled with a fluorophore, a radioisotope, or a non-isotopic labeling reagent such as biotin or digoxigenin.

In accordance with one embodiment known nucleic acid sequences, representing various genes of interest, are immobilized on a solid surface. Preferably the sequences are immobilized in the form of a microarray wherein each known sequence is assigned a position on a solid surface. In this manner a signal generated at a specific region of the solid surface by hybridization of a purified nucleosome DNA sequence to its complement identifies the gene encoded by that sequence. In one embodiment the purified nucleosome DNA is labeled (and in one embodiment the DNA is amplified and then labeled) and then placed in contact with a microarray of known sequences under conditions suitable for the hybridization of complementary sequences. After a predetermined length of time the unbound and non-specifically bound material is washed from the microarray and the array is screened for detectable signals.

The present invention also encompasses a method that utilizes an apoptosis marker for diagnosing disease states characterized by enhance cell death through apoptosis (e.g. cancer). It has been suggested that the presence of neoplastic cells in an individual will generate a higher level of nucleosomes in the blood as a result of apoptosis of such neoplastic cells. Accordingly, applicants anticipate that by limiting the analysis of nucleosomes to those released from apoptotic cells, the sensitivity of the diagnostic screen may be increased. A histone epitope has been identified (see International Patent Application PCT/US02/24405, the disclosure of which is incorporated herein) that serves as an apoptosis marker. An antibody directed against this epitope identifies cells that have been stimulated to enter an apoptotic death pathway with various artificial stimuli. This antibody is directed against the amino-terminal peptide Ser Ala Pro Ala Pro Lys Lys Gly Ser(P) Lys Lys (SEQ D NO: 7) of histone H2B (wherein “Ser(P)” represents a phosphorylated serine). This antibody can be used to selectively isolate nucleosomes that have been released from apoptotic cells.

Accordingly, in one aspect of the present invention this “apoptosis antibody” can be used to detect nucleosomes present in the bodily fluids of patients as a diagnostic indicator of diseases associated with apoptosis/enhanced cell death. Since the serine amino acid at the 14th position from the amino terminus (Ser14) of H2B is selectively phosphorylated in vivo in cells that will undergo or have already begun the process of apoptosis, this antibody may provide greater sensitivity for detecting nucleosomes present in blood that have been release from apoptotic cells of individuals that have, or are at risk of, developing a disease. Therefore this antibody may make a particularly effective diagnostic for detecting disease states in an individual. The diagnostic method comprises the steps of obtaining a blood, plasma or serum sample, contacting the sample with a composition comprising an antibody specific for a histone H2B amino terminal peptide that is phosphorylated at Ser14, such as the peptide Ser Ala Pro Ala Pro Lys Lys Gly Ser(P) Lys Lys (SEQ ID NO: 7), and immunoprecipitating nucleosomes bound to said antibody to recover those nucleosomes that were release from apoptotic cells. A threshold number of nucleosomes immunoprecipitated with the apoptosis marker antibody would be predictive of a disease state that is associated with apoptosis/enhanced cell death.

In accordance with one embodiment a method is provided for detecting tumor-related genes in a patient by analyzing the DNA associated with cell-free nucleosomes isolated from the patient. In accordance with this embodiment cell-free nucleosomes are isolated from a bodily fluid using one or more antibodies that specifically bind to histone proteins. The nucleosomes are immunoprecipitated and the associated DNA is purified and subjected to molecular analytical techniques to identify genes encoded by the purified DNA sequences. In one embodiment the DNA recovered from the immunoprecipitated nucleosomes is optionally amplified through PCR and then the DNA is contacted with a nucleic acid microarray, under conditions suitable for hybridization of complementary nucleic acid sequence, wherein the formation of nucleic acid duplexes produces a detectable signal. In this manner the genes encoded by the cell-free nucleosome associated DNA can be identified. Identifying the specific genes encoded by DNA associated with cell-free nucleosomes may have particular utility for monitoring the progress of a therapeutic treatment, including monitoring for positive effects as well as detecting adverse effects resulting from treatment. Furthermore, by selecting antibodies that target certain histone epitopes that are generated by post-translational modification of histone amino and carboxy tails, certain subsets of cell-free nucleosomes can be immunoprecipitated and the associated DNA analyzed.

In accordance with one embodiment a method is provided for detecting tumor-related genes or identifying fetal DNA in an adult female. The method comprises the steps of isolating DNA that is fetal or tumor in origin by taking advantage of a uniquely modified histone protein associated with the fetal or tumor gene of interest. For example, antibodies previously described in International Application No: PCT/US01/26283 specifically precipitate DNA associated with histones acetylated at lysine 9. Differential acetylation of histone associated with the gene of interest in the fetus relative to the maternal DNA will allow for the isolation and identification of the fetal DNA. In particular, the fetal DNA bearing nucleosomes can be selectively precipitated and the DNA recovered by PCR or other amplifying technique. Sequencing of the DNA recovered from the immunoprecipitated nucleosomes will allow the determination of the presence of absence of mutation in the fetal gene or nucleic acid sequences associated with tumor cells. Accordingly, this technique can serve as an important noninvasive technique for screening for genetic defects in fetuses as well as screening for early stage cancer.

In one important aspect of the present invention, the method can be used to determine if a fetus is heterozygous vs homozygous for a particular genetic defect. Therefore in one aspect, the present invention allows noninvasive prenatal diagnosis of genetic diseases and traits, having the advantage of being applicable even when the mother is a carrier of the condition. Furthermore, the prenatal diagnosis can be determined without the need for family studies.

In one embodiment the antibodies of the present invention are labeled. It is not intended that the present invention be limited to any particular detection system or label. The antibody may be labeled with a fluorophore, a radioisotope, or a non-isotopic labeling reagent such as biotin or digoxigenin; antibodies containing biotin may be detected using “detection reagents” such as avidin conjugated to any desirable label such as a fluorochrome. In one embodiment the histone specific antibodies of the present invention are detected through the use of a secondary antibody, wherein the secondary antibody is labeled and is specific for the primary (histone specific) antibody. Alternatively, the histone specific antibody may be directly labeled with a radioisotope or fluorochrome such as FITC or rhodamine; in such cases secondary detection reagents may not be required for the detection of the labeled probe. The presence of the modified histones in the blood can then be detected through the use of the relevant labeled antibody.

In accordance with one embodiment a method is provided for detecting chromatin alterations that are associated with a disease state. The method comprises the steps of isolating cell-free nucleosomes from biological samples taken from healthy individuals and from individuals afflicted with a disease to generate a first and second pool of nucleosomes, respectively. Typically, the biological sample will comprise a blood sample or derivative thereof (such as serum or plasma), however other bodily fluids that contain extracellular DNA can be used as well such as lymphatic fluid, urine, saliva. In one preferred embodiment the nucleosomes will be recovered from the biological sample through the use of one or more histone specific antibodies. In one embodiment the histone specific antibody is an antibody that binds to an epitope generated by one of the post-translational modifications of histone amino and carboxy tails indicated in FIG. 1. In one embodiment the histone specific antibody is an antibody that binds to a peptide comprising an amino acid sequence selected from the group consisting of

| | |
|---|---|
| Ala Arg Thr Lys(M) Gln Thr Ala Arg | (SEQ ID NO: 1), |
| Ser Gly Arg(M) Gly Lys, | (SEQ ID NO: 2), |
| Ser Gly Arg Gly Lys(A), | (SEQ ID NO: 3), |
| Ser Gly Arg(M) Gly Lys(A), | (SEQ ID NO: 4), |
| Ser(P) Gly Arg(M) Gly Lys(A), | (SEQ ID NO: 5), |
| Gln Thr Ala Arg Lys(M) Ser Thr Gly | (SEQ ID NO: 6), |
| Val | |
| Gln Thr Ala Arg Lys(M) Ser Thr Gly | (SEQ ID NO: 8) |
| Gly | |
| and | |
| Ala Ala Arg Lys(M) Ser Ala Pro | (SEQ ID NO: 9). |

In one embodiment the histone specific antibody is an antibody that binds to the peptide Ala Arg Thr Lys(M) Gln Thr Ala Arg (SEQ ID NO: 1) or Gln Thr Ala Arg Lys(M) Ser Thr Gly Gly (SEQ ID NO: 8).

After isolating the nucleosomes from the biological samples, the DNA associated with the isolated nucleosomes is purified from the first and second pools of nucleosomes to generate a first and second pool of purified DNA (representing a set of DNAs recovered from healthy individuals and a set of DNAs recovered from individuals suffering from a particular disease state). The purified DNAs are then analyzed, using standard molecular techniques such as DNA sequencing, nucleic acid hybridization analysis (including Southern blot analysis), PCR amplification or differential screening, to identify differences between the two pools of purified DNA sequences. Those nucleic acid sequences that are present in only one of the two pools of nucleic acid sequences represent expressed/suppressed genes (depending on the antibody used to isolate the nucleosomes) that are potentially related to the disease state.

In accordance with one embodiment the two pools of DNA recovered from the cell-free nucleosomes of healthy and non-healthy individuals are each separately contacted with identical sets of a DNA microarrays under conditions that allow for hybridization between complementary sequences. The microarrays may contain a subset of DNAs that are associated with particular diseases (such as various known oncogene and tumor suppressor genes) or it may contain the entire set of expressed sequences for one or more particular cell types and developmental stages. These known sequences can be immobilized on a solid surface or “chip” to form the microarray. Such microarrays can be prepared using techniques known to those skilled in the art. The microarrays are designed such that hybridization between a sequence in the nucleosome derived purified pool of DNA with a nucleic acid sequence of the microarray produces a detectable signal.

In one embodiment, the two pools of purified nucleosome DNA (i.e. from healthy and non-healthy sources) are labeled prior to contacting them with the microarray and in one embodiment the DNA sequences are amplified by PCR prior to labeling and contacting the sequences with the microarray. Subsequent washing of the array to remove non-bound and non-specifically bound material will allow detection of the labeled sequences that have specifically bound to the known sequence present on the microarray, thus revealing the identity of the labeled sequences. Furthermore, comparison of the hybridization pattern obtained with the first pool of purified DNA to the second pool of purified DNA reveals chromatin alterations that are potentially associated with a disease state.

Once a number of genes have been identified as being associated with a particular disease state, those gene sequences can then form basis for a diagnostic test using the present methodology. In accordance with one embodiment a method of screening/detecting a disease state comprises the steps of isolating cell-free nucleosomes from an individual through the use of antibodies specific for a modified histone, and determining the identity of nucleic acid sequences associated with the isolated nucleosomes. The identity of the sequences can be determined either by sequence analysis or by hybridization with known sequences. The identification of specific preselected nucleic acid sequences will be diagnostic for a disease state. For example, the presence of oncogenes or gene mutations that have been previously described as being associated with cancer may constitute the specific preselected sequence.

By combining chromatin immunoprecipitated DNA with current genomic microarray technology (on chips), one has the potential to survey any portion of the human (or other) genome relative to the unique modified histone associated with the sequence as it relates to the ‘histone code’. For example, DNA immunoprecipitated using the Methyl(K4)H3 antibody (specific for the sequence of SEQ ID NO: 1) can be immobilized on a solid surface or “chip” and thus represent all the nucleic acid sequences of a given cell that is competent for transcription. Similarly, DNA immunoprecipitated using the Methyl(K9)H3 antibody (specific for the sequence of SEQ ID NO: 6) can be immobilized on a solid surface or “chip” and thus represent all the nucleic acid sequences of a given cell that is not competent for transcription. Harvesting nucleosomes from the blood of an individual, recovering the associated DNA, labeling that DNA and then hybridizing the labeled DNA with the immobilized DNA microarrays will reveal abnormal expression of genes. The differences can be measured both qualitatively as well as quantitatively. Knowing this information may prove invaluable in determining the on/off state of key tumor suppressor or oncogenic proteins in various human cancers.

In one embodiment, immunoprecipitation of chromatin will be used to map the location of active genes at a genome-wide level through the use of microarrays. For example, in one preferred embodiment the method of comparing the two pools of immunoprecipitated chromatin (i.e. the immunoprecipitated chromatin from diseased vs healthy tissues) comprises the use of a gene chip, DNA microarray, or a proteomics chip using standard techniques known to those skilled in the art. For example any of the systems described in WO 01/16860, WO 01/16860, WO 01/05935, WO 00/79326, WO 00/73504, WO 00/71746 and WO 00/53811 (the disclosures of which are expressly incorporated herein) are suitable for use in the present invention. Preferably the chip will contain an ordered array of known compounds, such as known DNA sequences, so that interaction of the immunoprecipitated chromatin at a specific location of the chip will identify, and allow for the isolation of DNA sequences associated with the immunoprecipitated chromatin.

The key to this technology is the use of antibodies specific to various modifications as they relate to the histone code. Applying this to human and other genomes would lay the foundation of epigenomics. While the present invention has detailed the use of Lys4/Lys9 methyl H3 antibodies as respective ON/OFF antibodies, this concept applies more generally to any and all antibodies that are developed directed at the ‘histone code’. For example, Lys9 methyl vs. Ser10 phos H3 antibodies may also be a ‘methyl/phos’ switch that regulates differentiation vs. proliferation. The present invention also encompasses antibodies that are directed to other methylated regions of the amino terminus of H3 and H4 histones, including H3 lysines 27 and 36 and H4 lysine 20. The peptides that will be used to generate these antibodies are listed below:

| | | |
|---|---|---|
| H3 lysine 27: | AARK(M)SAPVCG | (SEQ ID NO: 10) |
| H3 lysine 36: | SGGVK(M)KPHKCG | (SEQ ID NO: 11) |
| H4 lysine 20: | RHRK(M)ILRDCG | (SEQ ID NO: 12) |

wherein K(M) represents a methylated lysine residue and underlined GC refers to amino acids added to the H3 sequence to aid in the production of this antibody.

### EXAMPLE 1

### Detection of Oligonucleosomes from Apoptotic Cells

MDA-MB468 human breast adenocarcinoma cells were seeded at 1.75×10⁶ cells/25 cm² in Leibovitz medium supplemented with 10% fetal calf serum. After 48 hrs, whilst in logarithmic growth, triplicate flasks of cells were stimulated with 0.1-0.3 M taxol or DMSO (vehicle, final concentration 0.002%) for 8 or 18 hrs at 37° C.

Cells were then gently rinsed with 2×10 ml of Dulbeccos phosphate buffered saline and then lysed for 30 minutes at room temperature in a minimal volume (200 l/25 cm²) of Lysis buffer:
Tris-HCl 50 mM, PH 7.4
NaCl, 150 mM
1% Triton-X100,
Sodium pyrophosphate, 2.5 mM
Glycerophosphate 10 mM
   This was supplemented immediately prior to use with an inhibitor cocktail designed to give final concentrations of:
EDTA, 10 mM
Trichostatin A, 200 ng/ml
Staurosporine, 2 M
Okadaic acid, 1 M
Cypermethrin, 0.5 M
AEBSF, 500 M (stable alternative to PMSF)
Aprotinin, 1 g/ml
E-64, 1 M
Leupeptin, 1 M
   Cell lysates were then centrifuged at 325 g for 10 minutes at 4° C., to pellet detergent insoluble (non-fragmented) chromatin, intact nuclei and cells. Supernatants, containing the enrichments of apoptotically generated mono and oligonucleosomes were harvested and stored in aliquots at −80° C. Samples were subsequently analyzed for nucleosome content by ELISA (Roche diagnostic kit: Cat No 1 774 425) and for the presence of ‘marked’ nucleosomes at the protein level by Western blotting using a series of antibodies, specific for phosphorylated, acetylated or methylated histones (Upstate).

The ELISA indicate an approximate 40-45 fold enrichment of nucleosomes in the 8 hr taxol treated cell supernatants relative to the vehicle. Maximal loadings of these nucleosome containing cell lysates (16 l/well) were resolved by electrophoresis on NuPAGE 12% Bis-Tris gels (reducing) using a MES buffer system (Novex). After transfer to nitrocellulose membranes the blots were probed overnight at 4° C., with polyclonal rabbit antibodies specific for methyl-histone H3 (lys-4), phosphorylated histone H3 (ser-10) and acetylated histone H3 (lys-14). Proteins were then visualized using an alkaline phosphatase conjugated anti-rabbit goat antibody in conjunction with a 5-bromo-4-chloro-3-indolyl-1-phosphate/nitro blue tetrazolium chromagenic substrate (Invitrogen). Positive results were obtained for both of these ‘histone-mark specific’ antibodies indicating that nucleosomes have retained phosphorylated, acetylated and methylated histone components during the apoptotic process.

1. A method of detecting active gene sequences in an individual, said method comprising the steps of:
providing a body fluid sample from said individual;
contacting the sample with an antibody that binds to a modified histone associated with active gene sequences;
isolating nucleosomes bound to said antibody;
purifying the DNA associated with said nucleosomes and
identifying a gene encoded by the purified DNA to detect a gene sequence that is active in said individual.

2. The method of claim 1 wherein the antibody specifically binds to a peptide selected from the group consisting of:

| | |
|---|---|
| Ala Arg Thr Lys(M) Gln Thr Ala Arg, | (SEQ ID NO: 1) |
| Ser Gly Arg(M) Gly Lys, | (SEQ ID NO: 2) |
| Ser Gly Arg Gly Lys(A), | (SEQ ID NO: 3) |
| Ser Gly Arg(M) Gly Lys(A), | (SEQ ID NO: 4) |
| and | |
| Ser(P) Gly Arg(M) Gly Lys(A). | (SEQ ID NO: 5) |

3. The method of claim 1 wherein the step of identifying a gene comprises sequencing the purified DNA.

4. The method of claim 3 wherein the purified DNA is PCR amplified before said sequencing step.

5. The method of claim 1 wherein the step of identifying genes comprises contacting said purified DNA with known nucleic acid sequences under conditions suitable for hybridization of complementary sequences, wherein hybridization of the purified DNA to its complement identifies the gene.

6. The method of claim 5 wherein the purified DNA is labeled prior to contacting the purified DNA with the known DNA sequences.

7. The method of claim 5 wherein the known nucleic acid sequences are immobilized on a solid surface.

8. The method of claim 1 wherein the bodily fluid is selected from the group consisting of urine, blood, lymph, plasma and serum.

9. The method of claim 7 wherein the body fluid is blood, plasma or serum.

10. A method of detecting inactive gene sequences in an individual, said method comprising the steps of:
providing a body fluid sample from said individual;
contacting the sample with an antibody that binds to a modified histone associated with inactive gene sequences;
isolating nucleosomes bound to said antibody;
purifying the DNA associated with said nucleosomes and
identifying a gene encoded by the purified DNA to detect a gene sequence that is inactive in said individual.

11. The method of claim 10 wherein the antibody specifically binds to a peptide selected from the group consisting of:

| | |
|---|---|
| Gln Thr Ala Arg Lys(M) Ser Thr Gly | (SEQ ID NO: 6) |
| Val, | |
| Gln Thr Ala Arg Lys(M) Ser Thr Gly | (SEQ ID NO: 8) |
| Gly, | |
| and | |
| Ala Ala Arg Lys(M) Ser Ala Pro. | (SEQ ID NO: 9) |

12. The method of claim 10 wherein the step of identifying a gene comprises sequencing the purified DNA.

13. The method of claim 12 wherein the purified DNA is PCR amplified before said sequencing step.

14. The method of claim 10 wherein the step of identifying the gene comprises contacting said purified DNA with known nucleic acid sequences under conditions suitable for hybridization of complementary sequences, wherein hybridization of the purified DNA to its complement identifies the gene.

15. The method of claim 14 wherein the purified DNA is labeled prior to contacting the purified DNA with the known nucleic acid sequences.

16. The method of claim 15 wherein the known nucleic acid sequences are immobilized on a solid surface.

17. The method of claim 16 wherein the known nucleic acid sequence comprises an immobilized DNA microarray and binding of the immobilized DNA with its complement produces a detectable signal.

18. The method of claim 10 wherein the body fluid is blood or sera.

19. A method of isolating nucleosomes released from apoptotic cells of an individual, said method comprising the steps of:
providing a blood or serum sample;
contacting the sample with a composition comprising an antibody that specifically binds to a peptide comprising the amino acid sequence Ser Ala Pro Ala Pro Lys Lys Gly Ser(P) Lys Lys (SEQ ID NO:7);
isolating nucleosomes bound to said antibody to recover those nucleosomes that were released from apoptotic cells.

20. A method for detecting chromatin alterations associated with a disease state, said method comprising the steps of:
isolating cell-free nucleosomes from samples taken from healthy individuals and individuals afflicted with a disease, through the use of antibodies specific for histone proteins, to generate a first and second pool of nucleosomes, respectively;
purifying the DNA associated with said isolated fist and second pools of nucleosomes to generate a first and second pool of purified DNA;
contacting the first and second pool of DNA with identical sets of a DNA microarrays under conditions that allow for hybridization between complementary sequences, wherein hybridization between a sequence in the purified pool of DNA with a DNA sequence of the microarray produces a detectable signal; and
comparing the hybridization pattern obtained with the first pool of purified DNA to the second pool of purified DNA to detect chromatin alterations associated with a disease state.

21. The method of claim 20 wherein the histone specific antibody specifically binds to a peptide selected from the group consisting of:

| | |
|---|---|
| Ala Arg Thr Lys(M) Gln Thr Ala Arg, | (SEQ ID NO: 1) |
| Ser Gly Arg(M) Gly Lys, | (SEQ ID NO: 2) |
| Ser Gly Arg Gly Lys(A), | (SEQ ID NO: 3) |
| Ser Gly Arg(M) Gly Lys(A), | (SEQ ID NO: 4) |
| Ser(P) Gly Arg(M) Gly Lys(A), | (SEQ ID NO: 5) |
| Gln Thr Ala Arg Lys(M) Ser Thr Gly | (SEQ ID NO: 6) |
| Val, | |
| Gln Thr Ala Arg Lys(M) Ser Thr Gly | (SEQ ID NO: 8) |
| Gly | |
| and | |
| Ala Ala Arg Lys(M) Ser Ala Pro. | (SEQ ID NO: 9) |

22. The method of claim 20 wherein said purified DNA is amplified prior to the step of contacting the DNA with the microarray.

23. The method of claim 20 wherein said purified DNA is labeled prior to the step of contacting the DNA with the microarray.

24. A method of detecting a disease state, said method comprising the steps of:
isolating cell-free nucleosomes from an individual through the use of an antibody specific for a modified histone; and
determining the identity of nucleic acid sequences associated with the isolated nucleosomes, wherein the identification of specific nucleic acid sequences is diagnostic for a disease state.

25. The method of claim 24 wherein the antibody specifically binds to a peptide comprising the amino acid sequences Ser Ala Pro Ala Pro Lys Lys Gly Ser(P) Lys Lys (SEQ ID NO:7).

26. The method of claim 24 wherein the nucleic acid sequences are identified by nucleic acid sequencing.
